(19) United States
(12) Patent Application Publication
(10) Pub. No.: US 2025/0000918 A1
(43) Pub. Date: Jan. 2, 2025

(54) METHODS AND COMPOSITION FOR THE TREATMENT OF PRADER-WILLI SYNDROME SYMPTOMS

(71) Applicant: The General Hospital Corporation, Boston, MA (US)

(72) Inventor: Xue-Jun Kong, Lexington, MA (US)

(73) Assignee: The General Hospital Corporation, Boston, MA (US)

(21) Appl. No.: 18/703,879

(22) PCT Filed: Oct. 25, 2022
(86) PCT No.: PCT/US2022/078622

§ 371 Date: Apr. 23, 2024

Related U.S. Application Data
(60) Provisional application No. 63/271,957, filed on Oct. 26, 2021.

(51) Int. Cl. A61K 35/745; A61K 9/00; A61K 35/00; A61P 1/14; C12Q 1/6874; C12Q 1/689

(52) U.S. Cl. CPC A61K 35/745; A61K 9/0053; A61P 1/14; C12Q 1/6874; C12Q 1/689; A61K 2035/115

(57) ABSTRACT

The present disclosure provides a method for treating symptoms of Prader-Willi Syndrome (PWS). The method includes administering a probiotic composition to the subject. The present disclosure further includes a. method, for determining an efficacy of a probiotic composition to treat PWS in a subject. The present disclosure further includes a kit. The kit includes a probiotic composition for oral administration and a detection molecule for the detection one or more microorganisms.

### CROSS-REFERENCE TO RELATED APPLICATIONS

This application claims priority to U.S. Provisional Patent Application No. 63/271,957 entitled “METHODS AND COMPOSITION FOR THE TREATMENT OF PRADER-WILLI SYNDROME SYMPTOMS” filed Oct. 26, 2021, the content of which is incorporated herein by reference in its entirety.

### STATEMENT REGARDING FEDERALLY SPONSORED RESEARCH OR DEVELOPMENT

Not Applicable

### BACKGROUND

Prader-Willi Syndrome (PWS) is an uncommon genetic syndrome that affects approximately one out of every 15,000 people. PWS is recognized as the most common genetic cause of life-threatening childhood obesity. Morbid obesity and neuropsychiatric complications are leading causes of death or long term disabilities. Besides some limited efficacy of growth hormone, the treatments are mainly behavioral. Accordingly, a non-behavioral treatment for PWS having with improved efficacy over current treatments is needed.

### SUMMARY

In one aspect, a method to alter the salivary microbiota of a subject in need is disclosed.

In embodiments, the method includes administering a probiotic composition to the subject.

In one or more embodiments, the probiotic composition includes Bifidobacterium animalis subsp. lactis (B. lactis).

In one or more embodiments, the probiotic composition includes BL-11.

In one or more embodiments, the subject in need thereof comprises a subject diagnosed with Prader-Willi Syndrome (PWS).

In another aspect, a method of determining an efficacy of a probiotic composition to treat PWS in a subject in need thereof is disclosed. In one or more embodiments, the method includes evaluating the salivary microbiota of the subject.

In another aspect, a kit is disclosed. In one or more embodiments, the kit includes a probiotic composition for oral administration.

In one or more embodiments, the kit includes a detection molecule for the detection of one or more of Faecalibacterium, Paracoccus, Leptotrichia, Bifidobacterium, Gemella, Aggregatibacter, Corynebacterium, Fusobacterium, Treponema, and Neisseria.

In some embodiments of the kit, the detection molecule includes an antibody or a nucleic acid.

### BRIEF DESCRIPTION OF DRAWINGS

FIG. 1 is a chart illustrating a timeline of a clinical study.

FIG. 2A is a set of graphs illustrating genus-level a diversity via Shannon Index over time for the salivary microbiome

FIG. 2B is a graph illustrating a Principal Coordinates Analysis (PCoA) of filtered fecal genera (right set of plots) and filtered salivary genera (left set of plots) using Bray-Curtis dissimilarity β-diversity post-treatment (i.e., weeks 6 and 12 combined). 95% confidence ellipses are shown.

FIG. 3A are network visualizations illustrating salivary co-abundance networks at weeks 0 (baseline, all subjects), 6, and 12 for each group. Nodes (circles) are sized and ordered concentrically by degree (i.e., number of connected edges).

FIG. 3B is a Venn diagram illustrating salivary taxa edges shared between groups and treatment status. Numbers in bold show the number of identified edges within the specified group and study timepoint.

FIG. 3C is a bar graph illustrating the number of chosen taxa in each network (e.g., total number of tax for each group as shown in FIG. 3B.

FIG. 4A is a set of graphs illustrating salivary Bifidobacterium increases over the study period in the active probiotic group.

FIG. 4B is a graph illustrating linear discriminant analysis effect size (LEfSe) intergroup comparisons of salivary microbiota relative abundance between groups following treatment (6- and 12-weeks combined).

FIG. 4C is a graph illustrating) GARS-3 cognitive ability score for different groups. The GARS-3 cognitive ability score is significantly negatively correlated with Paracoccus in subjects of ages 3 and older that are receiving the active probiotic following treatment (6- and 12-weeks combined).

FIG. 5 is a set of graphs illustrating correlations between differentially abundant salivary microbiota relative abundance and the predicted functional pathways in subjects receiving the active probiotic BL-11.

FIG. 6 is a set of graphs illustrating correlations between differentially abundant salivary microbiota relative abundance and the predicted functional pathways in subjects receiving the active probiotic BL-11.

### DETAILED DESCRIPTION

Previous clinical trials have shown that Bifidobacterium animalis subsp. lactis (B. lactis) improved anthropometric growth and behavioral severity in patient with Prader-Willi Syndrome (PWS). However, the effects of oral BL-11 supplementation on salivary microbiota composition have not yet been explored.

Salivary microbiome α diversity was found to be higher post-BL-11 supplementation at week 12 relative to placebo controls. Several differentially abundant microbiota were identified following BL-11 supplementation, including the genera Faecalibacterium, Paracoccus, Leptotrichia and Bifidobacterium (P<0.05). Several biological pathway gene abundances were found to be correlated with enriched bacteria in those receiving BL-11, suggesting associations with anti-inflammation, anti-obesity, toxin degradation, and anti-oxidative injury effects (P<0.05). Gemella, Aggregatibacter, Corynebacterium, Fusobacterium, and Treponema were found to be significantly correlated with height while Neisseria, Gemella and Paracoccus significantly correlated with social behaviors in the BL-11 treated group (P<0.05).

The inventors demonstrate herein that oral supplementation of BL-11 probiotic in individuals with PWS has the potential to induce favorable changes in the salivary microbiota composition through this post-hoc analysis. Characterization of the salivary microbiota following BL-11 supplementation has identified salivary microbiota signatures associated with height and socio-behavioral severity of PWS in the present study cohort.

The inventors anticipate that the findings of this study will shed light on the complex interactions between the salivary microbiome and the effects of the probiotic strain, as well as changes in aberrant behaviors and associated autism symptoms observed in individuals with PWS in response to probiotic supplementation. Furthermore, given the observed influences on the salivary microbiota following oral supplementation of the BL-11 probiotic in a powder format, it is of interest to assess the potential for further research and development of novel routes of administration for oral-use probiotics.

Oral microbiome, totally separated clusters from fecal microbiomes, also showed significant favorable changes in PWS patients following probiotics BL-11 intervention, these changes significantly correlated with their height and social behavioral improvement. These findings indicated that saliva sample, much easier assess than fecal sample, could serve as an independent biomarker or diagnostic tool to potentially screen and subtype PWS patients for corresponding further testing or treatments, and also could be used for monitoring treatment outcome as we found in this study. Additionally, the favorable changes in oral microbiome from probiotics BL-11 administration expanded the indication of this probiotics for improving oral health and added evidence of oral-gut-brain axis mechanism.

In embodiments, a method of altering the salivary microbiota of a subject in need thereof is disclosed. The method may be used in the altering of the salivary microbiota in any type of subject animal including but not limited to mammals, birds, reptiles, and amphibians. For example, the method may be used in the altering of the salivary microbiota of any type of mammal including but not limited to primates, rodents, bovines, ovines, porcines, equines, or any domesticated animal. For instance, the method may be use in the altering of salivary biota of any primate including but not limited to humans.

In embodiments, the method for altering the salivary microbiota in a subject may include any type of need, such as a treatment of a disease or a disease symptom, or an improvement in a subject characteristic. For example, the method may be used to treat genetic diseases and/or alleviate symptoms of genetic diseases. For instance, the method may be used in the treatment of, and/or alleviating one or more symptoms of, Prader-Willi Syndrome (PWS) in a subject diagnosed with PWS. In another example, the method may be used to treat diseases, or alleviate symptoms of diseases, having an environmental component including but not limited to cancer, diabetes, hypertension, cardiovascular disease, lung disease, and CNS diseases.

In embodiments, the method includes a step of orally administering a probiotic composition to the subject. The term probiotic as used herein refers to a substance, such as a live microorganism, which, when administered in adequate, or effective, amounts, confers a health benefit to the host. When referring to a live organism, the term “probiotic” may refer to the microorganism(s), or to the composition that includes the microorganism(s). A probiotic must fulfil several requirements related to lack of toxicity, viability, adhesion and beneficial effects. The term “effective amount” as used herein is an amount of probiotics which is sufficient to produce the expected effect.

In embodiments, the probiotic includes at least one microorganism. The at least one microorganism may be any organism that is adapted to live within at least one compartment of the digestive tract or alimentary canal including but not limited to the oral cavity (e.g., mouth,), oropharynx, esophagus, stomach, small intestine, and colon. For example, the microorganism may be an organism that commonly resides in the saliva of a subject.

In embodiments, the microorganism may include but not limited to a Bifidobacterium or a Lactobacillus genera of bacteria. For example, the microorganism may include at least one species of Bifidobacterium species or subspecies including but not limited to Bifidobacterium animalis, Bifidobacterium bifidum, Bifidobacterium longum, Bifidobacterium infantis, Bifidobacterium lactis, Bifidobacterium breve, and Bifidobacterium adolescentis. For instance, the microorganism may include any Bifidobacterium lactis bacterium, or any strain of Bifidobacterium lactis, such as the Bifidobacterium lactis BL-11 (e.g., a subspecies of Bifidobacterium animalis).

In embodiments, the probiotic may be included within a composition. For example, the composition may include one or more probiotics, such as a microorganism (e.g., Bifidobacterium lactis, such as the BL-11 strain). The composition may include one or more microorganisms. For example, the composition may include both Bifidobacterium and Lactobacillus strains. In another example, the composition may include two or more microorganism genera, species, or strains including but not limited to the microorganisms described herein.

In embodiments, at least one bacterial strain of Lactobacillus belongs to the species L. acidophilus, L. brevis, L. bulgaricus, L. casei, L. crispatus, L. delbrueckii, L. fermentum, L. gasseri, L. helveticus, L. lactis, L. plantarum, L. reuteri, L. rhamnosus, L. salivarius or L. paracasei. In still another embodiment, at least one bacterial strain of Lactobacillus species is L. acidophilus HA-122 (Lallemand Health Solutions (“LHS”)), L. acidophilus R0418 (LHS), L. brevis HA-112 (LHS), L. casei HA-108 (LHS), L. casei R0215 (LHS), L. delbrueckii bulgaricus HA-137 (LHS), L. fermentum HA-179 (LHS), L. helveticus HA-128 (LHS), L. helveticus HA-501 (LHS), L. helveticus R0052 (LHS), L. helveticus Lafti L10 R0419 (LHS), L. paracasei HA-196 (LHS), L. paracasei HA-274 (LHS), L. paracasei Lafti L26 R0422 (LHS), L. plantarum R0403 (LHS), L. plantarum R0202 (LHS), L. plantarum R1012 (LHS), L. reuteri HA-188 (LHS), L. rhamnosus HA-114 (LHS), L. rhamnosus HA-500 (LHS), L. rhamnosus R0011 (LHS), L. rhamnosus R0049 (LHS), L. rhamnosus R0343 (LHS), L. rhamnosus R1039 (LHS), L. salivarius HA-118 (LHS), L. salivarius R0078 (LHS), L. bulgaricus R0440 (LHS) or L. lactis R1087 (LHS).

In yet another embodiment, at least one bacterial strain of Lactobacillus belongs to the species L. rhamnosus. In still another embodiment, at least one bacterial strain of L. rhamnosus is L. rhamnosus HA-114 (LHS), L. rhamnosus HA-500 (LHS), L. rhamnosus R0011 (LHS), L. rhamnosus R0049 (LHS), L. rhamnosus R0343 (LHS) or L. rhamnosus R1039 (LHS). In an embodiment, at least one bacterial strain of L. rhamnosus is L. rhamnosus R0011 (LHS). In another embodiment, at least one bacterial strain of Bifidobacterium belongs to the species B. bifidum. B. animalis subsp. lactis, B. breve, B. longum, or B. longum subsp. infantis. In yet another embodiment, at least one bacterial strain of Bifidobacterium species is B. bifidum HA-132 (LHS), B. bifidum R0071 (LHS), B. breve HA-129 (LHS), B. breve R0070 (LHS), B. infantis HA-116 (LHS), B. infantis R0033 (LHS), B. lactis HA-194 (LHS), B. longum HA-135 (LHS), B. longum R0175 (LHS) or B. animalis subsp. lactis R0421 (LHS). In still another embodiment, at least one bacterial strain of Bifidobacterium species belongs to the species B. bifidum or B. longum. In an embodiment, at least one bacterial strain of B. bifidum is B. bifidum HA-132 (LHS) or B. bifidum R0071 (LHS).

In still another embodiment, at least one bacterial strain of B. bifidum is B. bifidum R0071 (LHS). In yet another embodiment, at least one bacterial strain of Bifidobacterium species is B. longum. In an embodiment, at least one bacterial strain of B. longum is B. longum HA-135 (LHS) or B. longum RO175 (LHS). In still another embodiment, at least one bacterial strain of B. longum is B. longum R0175 (LHS). In a further embodiment, the probiotic composition further comprises at least one microorganism strain belonging to a Streptococcus species, an Enterococcus species, a Lactococcus species, a Bacillus species or a Saccharomyces species. In yet another embodiment, the probiotic composition comprises at least one bacterial strain belonging to Lactobacillus species and Bifidobacterium species.

In an embodiment, at least one bacterial strain of Lactobacillus species belongs to the species L. rhamnosus and said at least one bacterial strain of Bifidobacterium species belongs to the species B. longum or B. bifidum. In still another embodiment, said at least one bacterial strain of Lactobacillus species belongs to the species L. rhamnosus and said at least one bacterial strain of Bifidobacterium species belongs to the species B. longum. In yet another embodiment, at least one bacterial strain of Lactobacillus species belongs to the species L. rhamnosus and said at least one bacterial strain of Bifidobacterium species belongs to the species B. bifidum. In an embodiment, said probiotic composition comprises at least one bacterial strain of L. rhamnosus, B. longum and B. bifidum.

In still another embodiment, at least one bacterial strain of L. rhamnosus is L. rhamnosus HA-114 (LHS), L. rhamnosus HA-500 (LHS), L. rhamnosus R0011 (LHS), L. rhamnosus R0049 (LHS), L. rhamnosus R0343 (LHS) or L. rhamnosus R1039 (LHS). In yet another embodiment, at least one bacterial strain of L. rhamnosus is L. rhamnosus R0011 (LHS). In still another embodiment, at least one bacterial strain of B. longum is B. longum HA-135 (LHS) or B. longum R0175 (LHS). In an embodiment, at least one bacterial strain of B. longum is B. longum R0175 (LHS). In an embodiment, at least one bacterial strain of B. bifidum is B. bifidum HA-132 (LHS) or B. bifidum R0071 (LHS). In an embodiment, at least one bacterial strain of B. bifidum is B. bifidum R0071 (LHS). In still another embodiment, the probiotic composition further comprises at least one microorganism strain belonging to the Streptococcus species, Enterococcus species, Lactococcus species, Bacillus species or Saccharomyces species.

In a further embodiment, at least one bacterial strain belonging to Lactobacillus species, Bifidobacterium species, or mixture thereof is for use at a dosage of from about 1×10⁵ to 1×10¹² cfu total bacteria per dose, from about 1×10⁶ to 1×10¹¹ cfu total bacteria per dose, from about 1×10⁷ to 1×10¹⁰ cfu total bacteria per dose or from about 1×10⁸ to 1×10¹⁰ cfu total bacteria cfu per dose.

The effective amount of colony forming units (“cfu”) for each strain in the composition will be determined by the skilled in the art and will depend upon the final formulation. The term “colony forming unit” is defined herein as number of bacterial cells as revealed by microbiological counts on agar plates. For instance, in an embodiment, the total probiotic is provided in an amount of from about 10⁵ to 10¹² colony forming units (cfu) per dose, from about 10⁶ to 10¹¹ cfu per dose, from about 10⁷ to 10¹⁰ cfu per dose or from about 10⁸ to 10¹⁰ cfu per dose. In yet another embodiment, the probiotic is provided in an amount greater than about 1.0×10⁹ cfu total probiotic per dose. In some embodiments, the individual is administered greater than 5.0×10⁹ cfu total probiotic per dose. However, it is not intended that the present disclosure be limited to a specific dosage as it is contemplated that dosages of total probiotic will vary depending upon a number of factors such as the identity and number of individual probiotic strains employed, the subject being treated, the nature of the symptoms suffered by the subject that is to be treated, the general health of the subject, and the form in which the composition is administered.

When a bacterium of the genus Bifidobacterium or a bacterium of the genus Lactobacillus is used in combination with one different probiotic microorganism (e.g., a different strain), the bacteria may be present in any ratio capable of achieving the desired effects of the disclosure described herein. Typically, the bacterial species or strains constituting the probiotic mixture are present in a mutual weight ratio of between about 100:1 to about 1:100, between about 50:1 to about 1:50, between about 20:1 to about 1:20, between about 10:1 to about 1:10, between about 9:1 to about 1:9, between about 8:1 to about 1:8, between about 7:1 to about 1:7, between about 6:1 to about 1:6, between about 5:1 to about 1:5, between about 4:1 to about 1:4, between about 3:1 to about 1:3, between about 2:1 to about 1:2 or 1:1.

While it is possible to administer the probiotics of the present disclosure alone, they are typically administered on or in a support as part of a product, in particular as a component of a food product, a dietary supplement, medicament or a pharmaceutical formulation. These products typically contain additional components, acceptable excipients, carriers or adequate additives well known to those skilled in the art. The term “acceptable excipients and carriers” as used herein pertains to those that are compatible with the other ingredients in the formulation and biologically acceptable. In a particular embodiment, the products additionally contain one or more further active agents. In another embodiment, the additional active agent or agents are other probiotic bacteria or yeasts which are not antagonist to the strains forming the composition of the present disclosure. Depending on the formulation, the strains may be added as purified bacteria, as a bacterial culture, as part of a bacterial culture, as a bacterial culture which has been post-treated. Prebiotics could be also added. The food product, the dietary supplement or the pharmaceutical formulation may be prepared in any suitable form which does not negatively affect the bioavailability of the strains forming the composition and is within the scope of ordinary persons skilled in the art.

For example, the probiotic composition of the present disclosure can be formulated to be administered orally, for ingestion, in the form of freeze-dried power, tablet, capsules, pills, suspension, lozenge, emulsion, liquid preparations, gel, syrup etc. The probiotic composition of the present disclosure can be used as an ingredient in food products such as milk products, yogurt, curd, cheese (e.g. quark, cream, processed, soft and hard), fermented milk, milk powder, milk based fermented product, ice-cream, a fermented cereal based product, milk based powder, a beverage, a dressing, meat products (e.g. liver paste, frankfurter and salami sausages or meat spreads), spreads, fillings, frostings, chocolate, confectionery (e.g. caramel, candy, fondants or toffee), baked goods (cakes, pastries), sauces and soups, fruit juices or coffee whiteners.

The probiotic microorganisms are produced by cultivating the microorganisms in a suitable medium and under suitable conditions as known in the art. The probiotic microorganisms can be cultivated alone to form a pure culture, or as a mixed culture together with other microorganisms, or by cultivating probiotic microorganisms of different types separately and then combining them in the desired proportions. After cultivation until a predetermined CFU/g concentration is reached, the cell suspension is recovered and used as such or treated in the desired manner, for instance, by concentrating, spray drying, lyophilization, flatbed oven drying or freeze-drying, to be further employed in the preparation of composition and can be blend with a carrier medium. Sometimes the probiotic preparation is subjected to an immobilization or encapsulation process in order to improve the shelf life. Several techniques for immobilization or encapsulation of bacteria are known in the art.

The probiotic strains used in the present disclosure are in the form of viable cells. However, the probiotic strains of the present disclosure can also be in the form of non-viable cells such as killed cultures or compositions containing beneficial factors produced by the probiotics. This could include thermally killed micro-organisms or micro-organisms killed by exposure to altered pH, sonication, radiation or subjection to pressure. With non-viable cells product preparation is simpler, cells may be incorporated easily into commercial products and storage requirements are much less limited than viable cells.

In accordance with the present disclosure, probiotic compositions will normally be administered so that a symptom-ameliorating effective daily dose is received by the subject. The daily dose may be given in divided doses as necessary, the precise amount of the compound or agent received and the route of administration depending on the general health of the subject being treated according to principles known in the art. A typical dosage regime is once, twice or three daily.

In embodiments, altering the salivatory microbiota of the subject includes increasing the α-diversity of the salivatory microbiota. The term “α-diversity” as used herein refers to the species diversity in a site at a local scale (e.g., such as within the oral cavity).

In embodiments, altering the salivatory microbiota of the subject includes altering the percentages of various microorganisms within the salivatory microbiota, introducing new microorganisms into the salivatory microbiota, and/or excluding microorganisms from the salivatory microbiota. The microorganisms affected by the altered salivatory microbiota may include microorganisms from several genera including but not limited to Faecalibacterium, Paracoccus, and Leptotrichia and Bifidobacterium, Fusobacterium, Corynebacterium, Gemella, Treponema, Neisseria, and Aggregatibacter genera. For example, altering the salivatory microbiota may include introducing or increasing the amount of Faecalibacterium, Paracoccus, Leptotrichia and/or Bifidobacterium after administration of the probiotic composition, as compared to an untreated control, or as compared to the subject prior to treatment. In another example, altering the salivatory microbiota may include introducing or increasing the amount of Gemella, Aggregatibacter, Corynebacterium, Fusobacterium, and/or Treponema after administration of the probiotic composition, as compared to an untreated control, or as compared to the subject prior to treatment.

In embodiments, the method may include an administration of the probiotic composition for any length of time. For example, the probiotic composition may be administered (e.g., as a course or regimen) for 1, 2, 3, 4, 5, 6, 7, 8, 9, 10, 11, 12, 13, 14, 15, 16, 20, 24, 28, 30, 34, 38, 42, 46, or 50 weeks or more. The probiotic composition may be administered monthly weekly, daily, or several times a day.

In embodiments, the administration of the probiotic composition to the subject, and/or the altered salivary microbiota of the subject is correlated with a change of or more characteristics/symptoms of the subject. Correlated changes may include increased height, improved social behavior, improved (e.g., lower) body max index (BMI), improved cognitive style (CS), improved emotional responses (ER), improved speech (e.g., decreased maladaptive speech (MS)), lower restricted/repetitive behaviors (RRB), increased social communication, and increased social interaction. As used herein, BMI is defined as a subject's weight in kilograms divided by the square of height in meters. Restricted/repetitive behaviors (RRB), social interaction (SI), social communication (SC), emotional response (ER), cognitive style (CS), and maladaptive speech (MS) may be evaluated by a clinician via the Gilliam Autism Rating Scale (GARS), such as the Third Edition of GARS (GARS-3).

In another aspect, a method of determining efficacy of the probiotic composition is disclosed. The method of determining efficacy of the probiotic composition my include evaluating the salivary microbiota of the subject, evaluating the symptoms of the disease/condition, and or evaluating characteristics of the subject. For example, the method may include determining evaluating the salivary microbiota of the subject, such as a subject having PWS. For instance, the method may include determining the presence or relative amount of one or more genera selected from: Faecalibacterium, Paracoccus, Leptotrichia, Bifidobacterium, Gemella, Aggregatibacter, Corynebacterium, Fusobacterium, Treponema, and Neisseria.

In embodiments, the evaluation of the salivary microbiota may include comparing the presence or relative amount to a control, with the control being either a non-treated subject (a second subject), or the salivary microbiota of the subject before treatment. The evaluation may occur at the end of the treatment, after the end of treatment or while the probiotic is being administered (e.g., at week 10 of a 12 week treatment). For example, evaluations may occur prior to probiotic treatment and 12 weeks after probiotic treatment.

In another aspect, a kit is disclosed that includes a probiotic composition, such as a probiotic composition for oral administration. For example, the kit may include the B. lactis, such as the B. lactis strain BL-11. The strain may also include one or more detection molecule for one or more microorganisms. For example, the kit may include detection molecules for various microorganisms including but not limited to the genera Faecalibacterium, Paracoccus, Leptotrichia, Bifidobacterium, Gemella, Aggregatibacter, Corynebacterium, Fusobacterium, Treponema, and Neisseria.

The detection molecule may be any type of molecule that can be used to distinguish microorganisms from each other. For example, the detection molecule may include an antibody that binds specifically to a genera, species, subspecies, or strain of a microorganism. The antibody may be labeled or capable of binding a labeled component (e.g., such as a secondary antibody) for detection. In this manner, the microorganisms may be identified and/or quantified via protein detection means (e.g., western blot or flow cytometry).

In another example, the detection molecular may include a nucleic acid, such an oligonucleotide pair, for amplification via polymerase chain reaction (PCR) of a region within the genome of the microorganism. The nucleic acid may be focused on specific genes or specific DNA sequences, such as the 16S rRNA gene. By isolating a heterogeneous sample saliva and amplifying/sequencing the 16S rRNA of the resultant isolated microorganisms, microorganisms may be detected and quantitated based on the sequences of the rRNA amplicons. The kit may also include genus-, species-, subspecies-, or strain-specific oligonucleotide pairs, wherein the presence of an amplicon indicates the presence of the microorganisms, without the need for sequencing.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Various embodiments and aspects of the present invention as delineated hereinabove and as claimed in the claims section below find experimental support in the following examples which, together with the above descriptions, illustrate some embodiments of the invention in a non-limiting fashion.

### Examples

Prader-Willi Syndrome (PWS) is a rare genetic disorder featuring by severe hypotonia and feeding difficulties in early infancy, and subsequent hyperphagia and early-childhood onset obesity. Additionally, developmental delay, short stature and numerous neuropsychiatric comorbidities have been implicated in individuals with PWS. (1-3). Gut microbiota has been implicated in the etiology of PWS subjects. (4) Previous studies have shown that the gut microbiome of individuals with PWS display patterns of dysbiosis. (5). Probiotics has shown improvement of metabolic disturbance and gut microbiome in PWS. (6,7).

We recently published two double blinded, randomized, and placebo-controlled clinical trials of probiotic supplementation for the treatment of anthropometric growth-associated comorbidities in PWS. (8,9) In our study on Bifidobacterium animalis subsp. lactis (BL-11) supplementation in individuals with PWS, we found a significant increase in height and improvement in CGI-I in those receiving the BL-11 probiotic relative to those receiving the placebo. Furthermore, the microbiota composition and metagenomic functional profiles also favored effects of weight loss and gut health with increased abundances of antioxidant production-related genes. (9).

The oral microbiome has been recognized as potential key biomarkers of several oral and systemic diseases. (10) In contrary of the well-studied gut microbiome, characterization of salivary microbiota composition and ecological diversity have not yet been explored in PWS populations. Furthermore, salivary microbiota-associated changes with probiotic intervention in PWS populations have not been previously reported. PWS patients were found to have high rate of oral diseases such as caries and tooth wear due to developmental delay, hyperphagia, and thick saliva. (11) Given that the human salivary microbiota is comprised of highly diverse groups of commensal, symbiotic, and pathogenic microorganisms. current research has suggested that such influences of the salivary microbiota extend beyond that of the oral cavity. (12) Several past studies have shown that oral microbiota possesses the ability to translocate to the gut with the potential to modulate the gut microbiome and host immune defense through the so-called microbiota-immune axis. (13) Current literature have also found that salivary microbiota may interact with the gut microbiome and impact brain function, suggesting that salivary microbiota may act as central mediators of the gut-brain communication (13,14). However, these areas of interests regarding the salivary microbiome, its relationship with core symptoms of PWS and gut microbiome composition, effects of longitudinal probiotic supplementation, and subsequent changes in the salivary microbiota in individuals with PWS have not been previously explored.

To fill these gaps of knowledge on the salivary microbiota in PWS, we performed the present post-hoc analysis based on our recently published clinical trial. (9) We aim to examine the oral microbiome profile in PWS patients, its changes following BL-11 probiotic intervention, and its associations with height growth, socio-behavioral symptom severity, and the relative levels of metagenomic functional pathways.

### Study Design

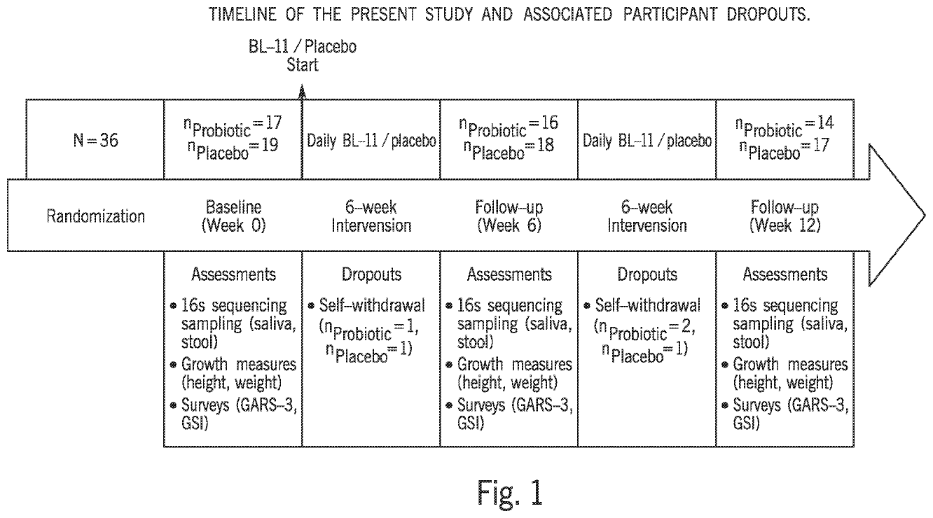

The original clinical trial design, protocol, randomization, blinding, participant eligibility, and intervention were well described in our previous publications. (8,9) The original clinical trial was registered under the Chinese Clinical Trial Registry with registration number ChiCTR1900022646 and involved 65 PWS subjects that were double-blinded and randomly assigned to either the probiotic interventional group or the placebo control group. (9) The enrolled subjects were subject to treatment for a total duration of 12 weeks. In this post-hoc analysis study, we included a subset of 36 subjects with ages 59.49±40.56 months who had available salivary sample 16s sequencing data. Among the subset of 36 subjects, 17 subjects (aged 60.66±32.19 months) were allocated to the probiotic group and 19 subjects (aged 58.5±47.34 months) were allocated to the placebo group. Probiotics BL-11 (Beijing Huayuan Academy of Biotechnology) was used in the study in the format of a sachet containing the probiotic BL-11 in powder form. Each sachet of probiotics supplement contained 3×10¹⁰ colony forming units (CFUs). The placebo was maltodextrin in the sachet with similar color, flavor, and taste as the probiotic sachets. Subjects received one sachet twice a day of either probiotic or placebo for a duration of 12 weeks and were instructed to consume the sachet contents orally with water. No adverse events were observed throughout the study course. An illustration of the timeline, sample sizes, and participant dropouts are shown in FIG. 1.

### Outcome Measurements and Data Collection

Outcome measurements were taken at weeks 0 (baseline), 6, and 12. Weight and height measurements were measured by parents using standard scales and recorded by the research staff for all enrolled subjects regardless of age. Restricted/repetitive behaviors (RRB), social interaction (SI), social communication (SC), emotional response (ER), cognitive style (CS), and maladaptive speech (MS) were evaluated by an experienced clinician via the Gilliam Autism Rating Scale, Third Edition (GARS-3) for those of ages 3 years or older (15). Furthermore, medical, dental, and dietary histories were recorded during the visits.

DNA Extraction and 16S rRNA Sequencing of Saliva Samples

Bacterial genomic DNA was extracted from saliva samples using the Powersoil DNA isolation kit (Qiagen, Duesseldorf, Hilden, Germany) with the bead-beating method according to the manufacturer's instructions. Characterization of the salivary microbiota was done via high fidelity 16S rRNA amplicon gene sequencing based on collected saliva samples of all subjects of this study.

### Bioinformatics Processing of Amplicon Sequencing Data

Sequencing reads were bioinformatically processed using Biobakery Workflows (v0.13.2) (16) based on the VSEARCH (v2.14.1) (17) method. In short, the sequences were demultiplexed and VSEARCH was used with default parameters to merge, filter, and trim the Illumina data. The sequences were then dereplicated, sorted by size, and clustered into operational taxonomic units (OTUs). Next, phylogenetic trees were constructed after alignment of sequences using Clustal Omega. The taxonomies of OTUs were assigned using the Greengenes database (v13.8) with sequences sharing 97% similarity. (18) The resulting reads were transformed via total sum scaling and filtered using a prevalence threshold of 0.0001 and an occurrence threshold of 10% of the population.

### Statistical Analysis

All raw data were recorded and processed in Microsoft Excel 2016 and R. Statistical procedures were carried out using α=0.05 as the significance level. Data analysis and visualization was performed under R using the ggplot2 package while statistics were generated using the compatible ggpubr package. Univariate linear correlations via MaAsLin2 was used to explore per-feature correlations between clinical indices, predicted functional profiling results and microbial taxa abundances. Resulting P-values were adjusted for multiple testing using false-discovery rate (FDR) based on the Benjamini-Hochberg method. (19) Complete genus-level salivary microbiota co-abundance network analyses were performed using the NAMAP with Spearman's rank correlation algorithm while applying a significance cutoff of α=0.05 with 100 bootstrap iterations via MetagenoNets. (20)

### Results

### Baseline Subject Demographics and Clinical Indices

We included 36 subjects aged 59.49±40.56 months (52.78% male, 47.22% female) with genetically confirmed diagnosis of Prader-Willi syndrome. Of which, 17 subjects aged 60.66±32.19 months were randomized to receive the active probiotic, while 19 subjects aged 58.5±47.34 months were randomized to receive placebo for a duration of 12 weeks. Of 36 subjects included in this study, there were 29 of them had available GARS-3 dataset (GARS-3 is only applicable for those age 3 or above), 17 subjects were in placebo group and 12 subjects were in probiotics group. No adverse events were reported during the trial period. A summary of the subject demographics and detailed clinical indices are provided in Table 1, which indicated no significant differences between the probiotic and placebo groups among all the demographic and clinical parameters as listed.

TABLE 1
Summary of baseline subject demographics and measured clinical indices.

| | | Placebo | Probiotic | χ²-test | Overall |
|---|---|---|---|---|---|
| | | (n = 19) | (n = 17) | P-value | (n = 36) |
| Sex (n) | Male | 10 (52.63%) | 9 (52.94%) | 0.99 | 19 (52.78%) |
| | Female | 9 (47.37%) | 8 (47.06%) | | 17 (47.22%) |

| | | | | | | | | | Mann- | | | | |
|---|---|---|---|---|---|---|---|---|---|---|---|---|---|
| | | | | | | | | | Whitney | | | | |
| | | | | | | | | | U test | | | | |
| | Mean (SD) | | Median (IQR) | | Mean (SD) | | Median (IQR) | | P-value | Mean (SD) | | Median (IQR) | |
| Age (month) | 58.5 | (47.34) | 40.5 | (53) | 60.66 | (32.19) | 58.5 | (43.38) | 0.48 | 59.49 | (40.56) | 46.5 | (48.25) |
| Height (cm) | 111.25 | (20.71) | 112.5 | (33.25) | 107.4 | (21.37) | 112 | (33) | 0.81 | 109.11 | (19.83) | 112 | (37) |
| Weight (kg) | 26.45 | (15.29) | 26.65 | (25.35) | 29.8 | (20.02) | 29 | (24) | 0.90 | 28.31 | (17.06) | 29 | (24.8) |
| Body mass | 19.47 | (5.04) | 19.55 | (8.49) | 23.01 | (9.07) | 18.86 | (13.39) | 0.73 | 21.43 | (7.36) | 18.86 | (8.55) |
| index (BMI) | | | | | | | | | | | | | |
| GARS-3 | n = 17 | | | | n = 12 | | | | | n = 29 | | | |
| Cognitive | 10 | (3.61) | 11 | (3.5) | 8.67 | (2.08) | 8 | (2) | 0.82 | 9.33 | (2.73) | 9.5 | (3.75) |
| style (CS) | | | | | | | | | | | | | |
| Emotional | 10.33 | (3.21) | 9 | (3) | 15.33 | (4.04) | 13 | (3.5) | 0.38 | 12.83 | (4.26) | 13 | (3.75) |
| responses (ER) | | | | | | | | | | | | | |
| Maladaptive | 10 | (5.29) | 12 | (5) | 7.33 | (3.79) | 9 | (3.5) | 0.40 | 8.67 | (4.37) | 9.5 | (6.25) |
| speech (MS) | | | | | | | | | | | | | |
| Restricted/ | 19 | (6) | 19 | (6) | 24.33 | (9.29) | 20 | (8.5) | 0.70 | 21.67 | (7.58) | 19.5 | (5.5) |
| Repetitive | | | | | | | | | | | | | |
| behaviors | | | | | | | | | | | | | |
| (RRB) | | | | | | | | | | | | | |
| Social | 19.33 | (6.81) | 17 | (6.5) | 12.33 | (7.57) | 9 | (7) | 0.40 | 15.83 | (7.49) | 15.5 | (9.75) |
| communication | | | | | | | | | | | | | |
| (SC) | | | | | | | | | | | | | |
| Social | 10.67 | (2.52) | 11 | (2.5) | 9.33 | (8.02) | 10 | (8) | 1.00 | 10 | (5.37) | 10.5 | (4) |
| interaction (SI) | | | | | | | | | | | | | |

### Global Salivary Microbiome Biodiversity and Co-Abundance Network Changes Following BL-11 Supplementation

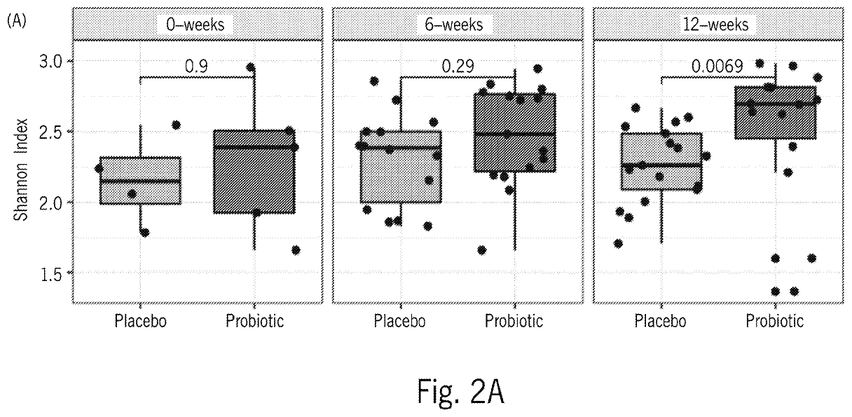

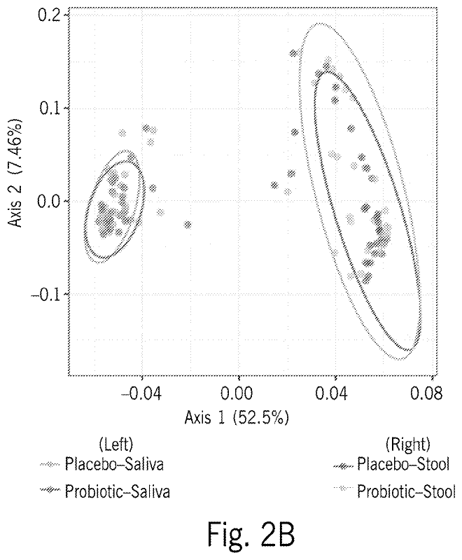

Changes in the salivary microbiome biodiversity were assessed through intergroup comparisons of mean a diversity at weeks 0, 6, and 12. An increasing trend in Shannon index was observed over the 12-week study period for the active probiotic group and a significant groupwise difference in mean Shannon index is observed at week 12 (FIG. 2A, P<0.05). β diversity of both salivary and fecal microbiomes following treatment (including samples from weeks 6 and 12 combined) are illustrated in FIG. 2B and the corresponding 95% confidence ellipses are illustrated by treatment group and sample source.

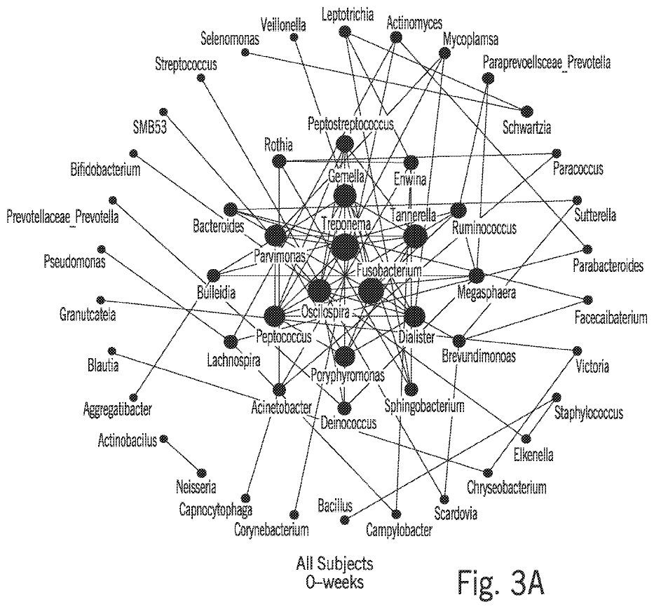

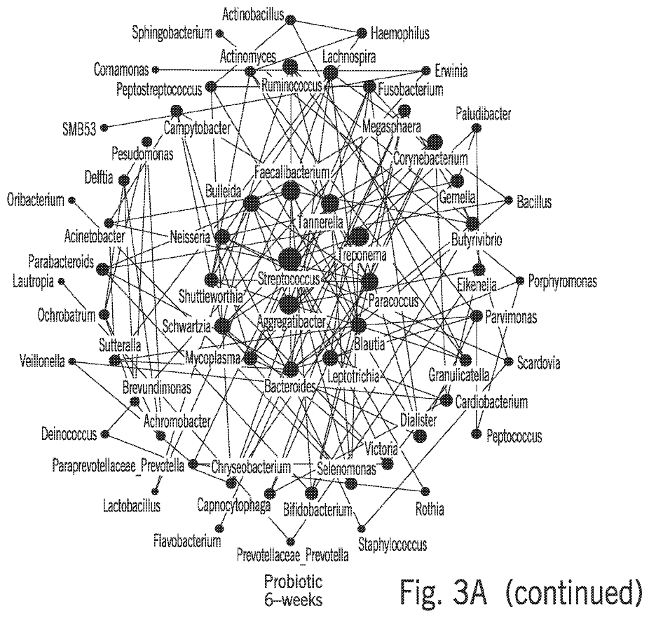

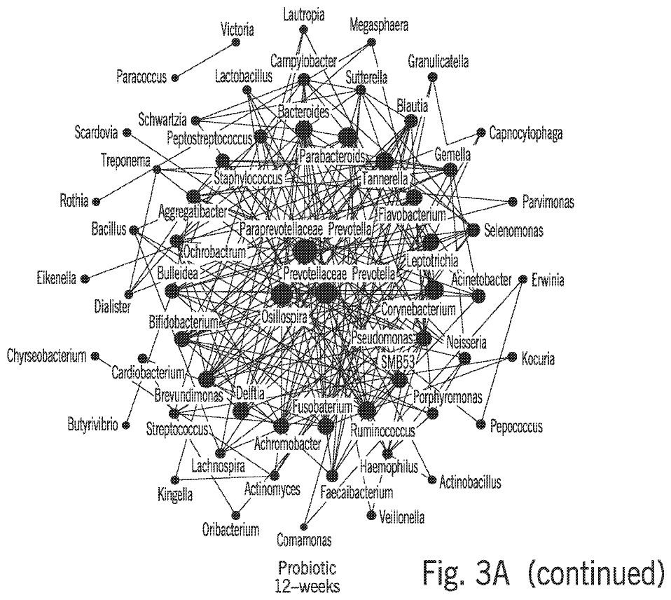

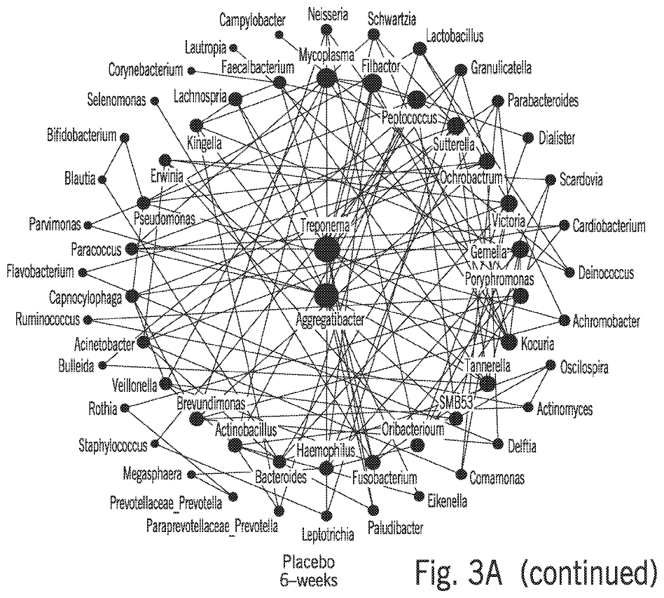

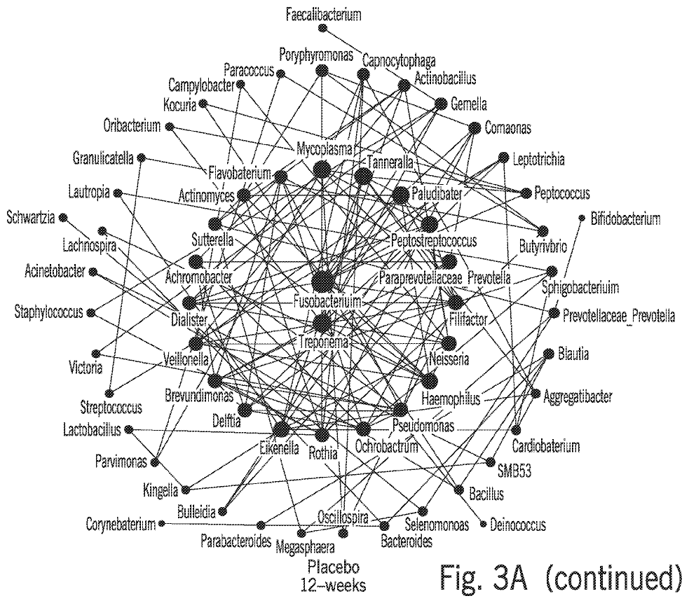

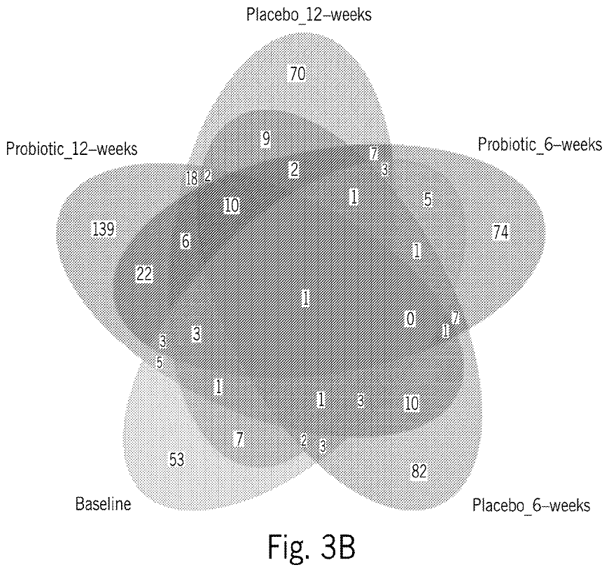

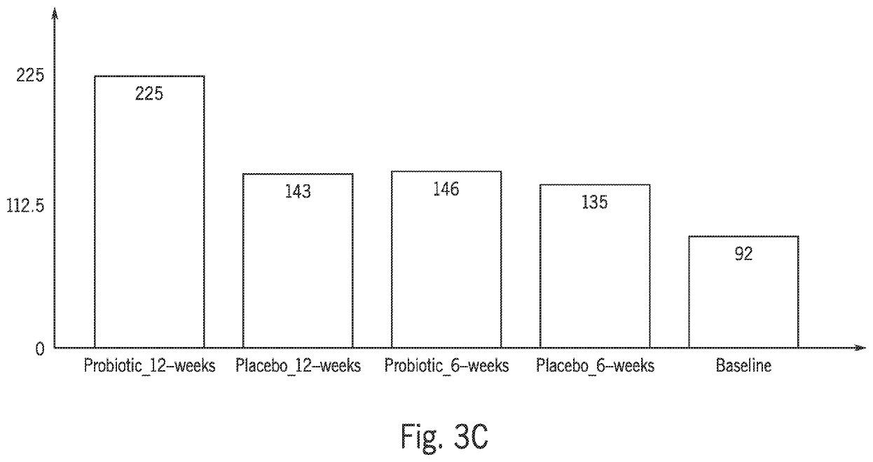

To assess the interactions between genus-level salivary microbiota relative abundance by group per each study visit, we constructed co-abundance networks based on the NAMAP with Spearman's rank correlation algorithm as shown in FIG. 3A. An overview of the number of unique and shared edges within each group based on the treatment status are shown in FIG. 3B, with total number of edges (e.g., taxa) identified in each group shown in FIG. 3C.

### Altered Genus-Level Salivary Microbiota Following BL-11 Supplementation

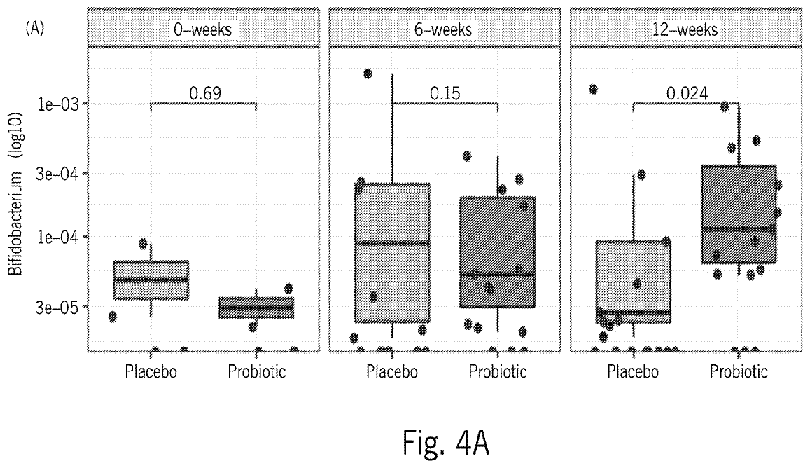

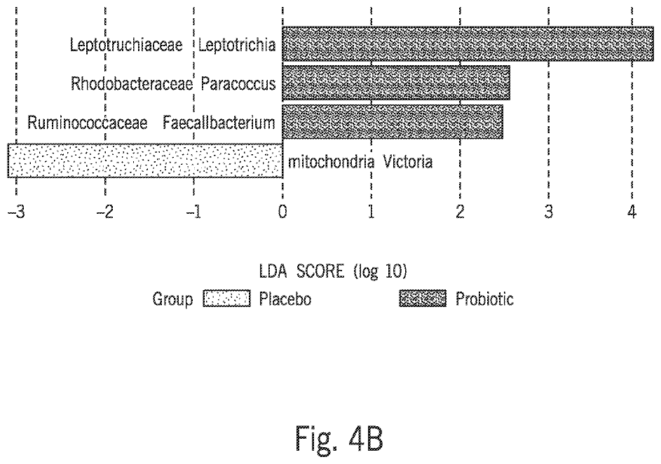

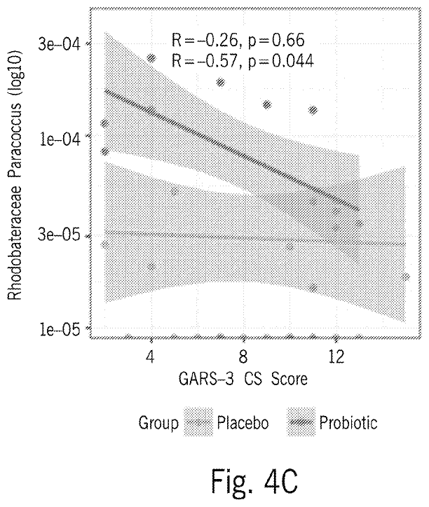

To assess for the salivary microbiota abundance changes over the course of probiotic supplementation, we performed groupwise comparisons of genus-level microbiota relative abundances for specific genera of interest while overall microbiota changes following treatment was assessed via linear discriminant analysis effect size (LEfSe). The Bifidobacterium genus relative abundance displayed an increasing trend over the treatment course in the active probiotic group and showed significant intergroup differences in relative abundance at week-12 (FIG. 4A, P<0.05). Globally, the Leptotrichia, Paracoccus, Mycoplana, and Bifidobacterium genera were significantly more abundant in the active probiotic group salivary microbiome, whereas the Victoria genus (Mitochondria) was significantly more abundant in subjects receiving placebo using data from weeks-6 and -12 combined (FIG. 4B, P<0.05). Among the differentially abundant genera determined via LEfSe in the active probiotic group, Paracoccus relative abundance was found to be negatively correlated with GARS-3 cognitive style (CS) score (FIG. 4C, P<0.05) while other differentially abundant microbiota were not found to be significantly correlated with socio-behavioral severity scores.

### Associations Between Post-Treatment Socio-Behavioral Severity, Height, Weight, Predicted Functional Pathways, and Salivary Microbiota Abundance.

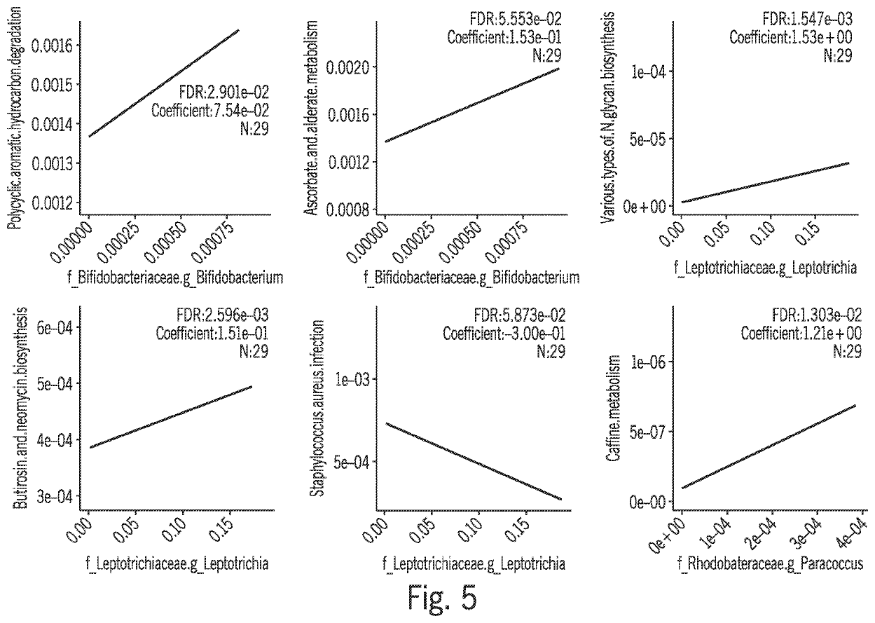

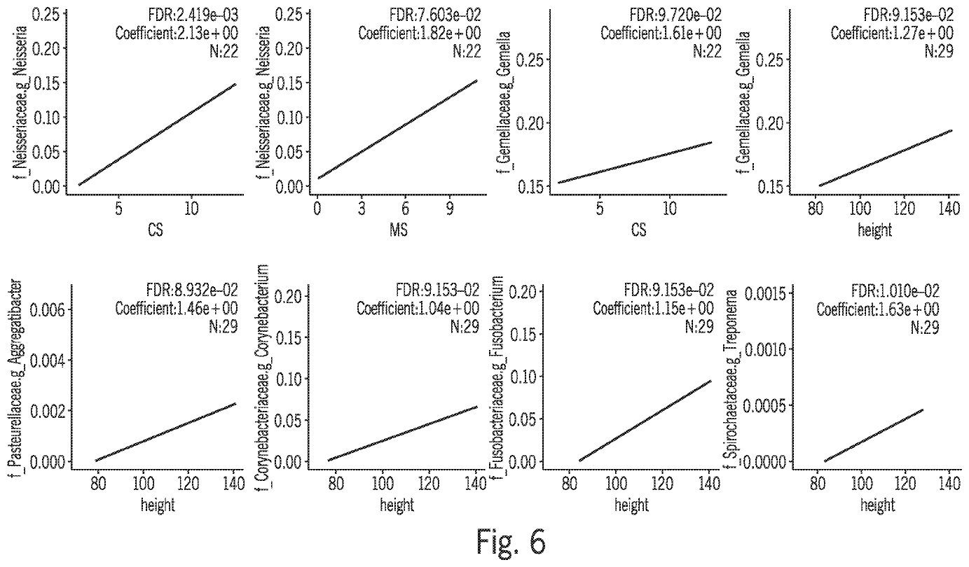

In an attempt to elucidate the functional role of differentially abundant microbiota and determine the relationships between salivary microbiota relative abundance, socio-behavioral symptom severity, weight, and height following active probiotic supplementation, we performed linear regression with false discovery rate adjustments to the P-values to account for multiple comparisons. Statistical significance was considered by applying a significance cutoff of FDR<0.1 and the resulting correlations are presented in FIGS. 5 and 6.

### Discussion

In the present study, we explored and compared salivary microbiota profiles before and after supplementation with the probiotic B. lactis BL-11 in individuals with PWS through a post-hoc analysis. We found that the PCoA of Bray-Curtis dissimilarity β diversity shows defined, separated clusters between salivary and fecal microbiomes both before and after intervention in the two groups (i.e., receiving either BL-11 or placebo), which is suggestive of inter-microbiome differences in ecological diversity and is consistent with our expectations. However, the separation in sample clusters were not identified between groups of the same sample source, which may suggest that oral supplementation of BL-11 does not globally alter the salivary and fecal microbiome composition when considering the Bray-Curtis dissimilarity metric in terms of β diversity. Conversely, the Shannon index as a measure of a diversity was found to show an increasing trend over the treatment course for those receiving the BL-11 probiotic and is significantly different between groups at week-12. Such results suggest that while oral supplementation of BL-11 does not uniformly alter the global salivary microbiome composition relative to those receiving the placebo, subjects receiving the BL-11 probiotic independently exhibit more heterogeneity in the salivary flora. Using genus level co-abundance networks, we observed higher numbers of edges in the post-BL-11 treatment group at week-12. We hypothesize that such effects are associated with the administration of the BL-11 probiotic. Based on our findings, we observed an increasing trend and statistically significant higher abundances of salivary Bifidobacterium following BL-11 treatment relative to those receiving the placebo. The change in salivary Bifidobacterium relative abundance at week-12 is consistent with expectations due to the method of probiotic delivery; as the probiotic was administered orally in a powder format, we suspect that the exposure of the BL-11 probiotic within the oral cavity led to the increase in oral Bifidobacterium over time. Furthermore, we found that subjects receiving the BL-11 probiotic intervention have higher abundances of several bacterial genera, including Faecalibacterium, Paracoccus, and Leptotrichia, relative to subjects receiving the placebo. Taken together, these findings suggest that BL-1l supplementation can induce specific compositional changes in the salivary microbiota following a 12-week interventional period.

Given our current understanding of the multidirectional interactions between the host immune system, brain, gut, and microbiota (13,21), we consider the observed higher abundance of Faecalibacterium post-BL-11 treatment as a potentially beneficial effect that would likely influence socio-behavioral symptoms and anthropometric growth in individuals with PWS. Faecalibacterium prausnitzii is the sole known species belonging to the Faecalibacterium genus and has been known to have butyrogenic effects within the gut microbiome. (22) A growing body of literature have suggested that the production of short chain fatty acids (SCFAs) within the gut possess anti-inflammatory effects, with acetate, propionate, and butyrate being the most abundant products. (22,23) Mechanistic studies have demonstrated SCFA activation of mammalian G protein-coupled receptors (GPCRs) GPR41 and GPR43 in vitro. (24,25) Furthermore, studies in mice have suggested that such a mechanism underlies the anti-inflammatory (26) and anti-obesity (27) effects in response to SCFA in the gut; however, there remains a large degree of heterogeneity in relevant findings and further research in this field is warranted. (28) Nonetheless, clinical studies in patients with Type 2 Diabetes (T2D) have implicated lower levels of butyrogenic fecal microbiota, including Faecalibacterium, and gut microbiome dysbiosis, which may present relevance to individuals with PWS due to its prevalence in PWS as a comorbidity (29).

In contrast to the well-studied Faecalibacterium, the properties of the Paracoccus genus remains largely unknown, though it has been previously identified in the skin flora (30), which may implicate aberrant behavioral patterns in PWS as a potential cause of the inhabitance of such microbiota within the salivary microbiome. Interestingly, we identified a significant negative correlation between the relative abundance of a Paracoccus species and GARS-3 cognitive style score among subjects receiving the BL-11 probiotic while this trend was not found to be statistically significant among subjects receiving the placebo. We postulate that the higher salivary abundance of Paracoccus relative to the controls and negative correlation with GARS-3 cognitive style score post-intervention is mediated through the oral-gut-brain axis due to the introduction of the BL-11 probiotic. Additionally, in the metagenomic analysis of predicted functional pathways. we found that the Paracoccus species was positively correlated with caffeine metabolism in those receiving the BL-11 probiotic. Caffeine was found to help fat utilization and reduction of obesity. (31,32) Leptotrichia species exist as part of the normal flora of the human oral cavity and was found to be positively correlated with the biosynthesis of N-glycans, neomycin metabolism, and negatively correlated with Staphylococcus aureus infection post-BL-11 treatment.

As past studies have suggested, the importance of glycan expression in the bidirectional microbiota-host and inter-microbiota interactions within the oral microbiome for promoting host oral health and defense (33), these findings may suggest that the increase in Leptotrichia post-BL-11 intervention is a beneficial alteration to individuals with PWS in preventing oral infections from pathogenic species. Similarly, the analysis of predicted functional pathways from the salivary metagenome indicated that salivary Bifidobacterium was found to be significantly positively correlated with Vitamin C metabolism and degradation of Polycyclic Aromatic Hydrocarbons (PAH). Vitamin C is an antioxidant and have been suggested to promote host defense against periodontal diseases and promote general tooth and gingivae health. (34) Moreover, the positive correlation between Bifidobacterium abundance and degradation of PAH may suggest a role of Bifidobacterium in the oral metabolism and clearance of PAHs. PAHs have been characterized as pervasive environmental and dietary toxicants and carcinogens. (35) Existing literature has suggested that the toxicity of PAHs is associated with its estrogenicity within the human colon following biotransformation of unabsorbed PAHs due to colonic microbiota. (36) Given the findings within the current study, the positive correlation between PAH degradation and Bifidobacterium relative abundance observed in oral saliva samples is suggestive of the potential for BL-11 supplementation to decrease the levels of unabsorbed PAHs reaching the colon, thereby reducing the likelihood of PAH-associated toxicity in subjects receiving the BL-11 probiotic.

In our assessment of microbiota associations against socio-behavioral severity and anthropometric growth measures, Neisseria was found to be positively correlated with both GARS-3 cognitive style and maladaptive speech scores while Gemella was found to be positively correlated with only maladaptive speech score following BL-11 supplementation. Provided that we have previously found compositional differences in both salivary and fecal microbiota between individuals with ASD and healthy controls (37), recent literature has further identified associations between several mental disorders and oral microbiota dysbiosis, thereby supporting our hypothesis of oral microbiota-brain interactions. (38) Thus, such findings may implicate signatures of oral microbiota compositional alterations in individuals with PWS following probiotic treatment, though the causal relationship remains to be validated in future studies. Importantly, our published clinical trial of BL-11 has found that height was found to be significantly increased following BL-11 intervention (9).

In this study, we found that several salivary microbiota were significantly positively correlated with height post-BL-11 treatment, including the genera Gemella, Aggregatibacter, Corynebacterium, Fusobacterium, and Treponema, while such correlation were not statistically significant in those receiving the placebo. Given our current understanding, the identified height-correlated genera in the present study have been described in literature as largely non-pathogenic genera and are commonly found within the oral microbiome (10,39,40). We postulate that these oral taxa may play a role in the mediation of childhood growth, specifically in terms of height. In a study conducted by Vonaesch et al., the over-representation of oropharyngeal microbiota within the gut has been proposed to be associated with stunted growth in African children aged 2 to 5 years old. (41) Among the several over-represented oropharyngeal microbiota in the gut of children with stunted growth identified by Vonaesch et al., the genera Gemella, Fusobacterium, and Aggregatibacter were found to be positively correlated with height post-BL-11 treatment in the present study. Taken together, we propose that these specific salivary microbiota signatures may represent valuable features in the early diagnosis of stunted anthropometric growth in children with PWS. Furthermore, due to the non-invasive nature of the sample collection, salivary microbiota sampling is likely preferred over fecal microbiota sampling, provided that the application of such a technique can demonstrate sufficient sensitivity and specificity in classification through future studies.

In the present study, we demonstrate that oral supplementation of BL-11 probiotic in individuals with PWS has the potential to induce favorable changes in the salivary microbiota composition through this post-hoc analysis. Characterization of the salivary microbiota following BL-11 supplementation has identified salivary microbiota signatures associated with height and socio-behavioral severity of PWS in the present study cohort. However, due to the number of dropouts, small sample size, and a homogenously Chinese study population, the results should be interpreted with caution. We hope that the findings of this study could shed light on the complex interactions between the salivary microbiome and the effects of the probiotic strain, as well as changes in aberrant behaviors and associated autism symptoms observed in individuals with PWS in response to probiotic supplementation. Furthermore, given the observed influences on the salivary microbiota following oral supplementation of the BL-11 probiotic in a powder format, it is of interest to assess the potential for further research and development of novel routes of administration for oral-use probiotics.

This study was conducted according to the guidelines of the Declaration of Helsinki and approved by the Institutional Review Board of the Second Affiliated Hospital of Kunming Medical University (Review-YJ-2016-06, Feb. 21, 2019). Informed consent was obtained from all subjects involved in the study. The data presented in this study are openly available in the Sequence Read Archive (SRA) database of The National Center for Biotechnology Information at https://www.ncbi.nlm.nih.gov/bioproject/643297, with accession number PRJNA643297.

### LIST OF REFERENCES

1. Cassidy S B, Forsythe M, Heeger S, Nicholls R D, Schork N, Benn P, Schwartz S. Comparison of phenotype between patients with Prader-Willi syndrome due to deletion 15q and uniparental disomy 15. Am J Med Genet (1997) 68:433-440. doi:10.1002/(sici)1096-8628(19970211)68:4<433::aid-ajmg12>3.0.co;2-t
2. Butler M G, Hartin S N, Hossain W A, Manzardo A M, Kimonis V, Dykens E, Gold J A, Kim S-J, Weisensel N, Tamura R, et al. Molecular genetic classification in Prader-Willi syndrome: a multisite cohort study. J Med Genet 56:149-153. doi:10.1136/jmedgenet-2018-105301
3. Salehi P, Herzig L, Capone G, Lu A, Oron A P, Kim S. Comparison of Aberrant Behavior Checklist profiles across Prader-Willi syndrome, Down syndrome, and autism spectrum disorder. Am J Med Genet A (2018) 176:2751-2759. doi:10.1002/ajmg.a.40665
4. Olsson L M, Poitou C, Tremaroli V, Coupaye M, Aron-Wisnewsky J, Bäckhed F, Clément K, Caesar R. Gut microbiota of obese subjects with Prader-Willi syndrome is linked to metabolic health. Gut (2020) 69:1229-1238. doi:10.1136/gutjnl-2019-319322
5. Peng Y, Tan Q, Afhami S, Deehan E C, Liang S, Gantz M, Triador L, Madsen K L, Walter J, Tun H M, et al. The Gut Microbiota Profile in Children with Prader-Willi Syndrome. Genes-basel (2020) 11:904. doi:10.3390/genes11080904
6. Ke X, Walker A, Haange S-B, Lagkouvardos I, Liu Y, Schmitt-Kopplin P, Bergen M von, Jehmlich N, He X, Clavel T, et al. Synbiotic-driven improvement of metabolic disturbances is associated with changes in the gut microbiome in diet-induced obese mice. Mol Metab (2019) 22:96-109. doi:10.1016/j.molmet.2019.01.012
7. Hibberd A A, Yde C C, Ziegler M L, Honoré A H, Saarinen M T, Lahtinen S, Stahl B, Jensen H M, Stenman L K. Probiotic or synbiotic alters the gut microbiota and metabolism in a randomised controlled trial of weight management in overweight adults. Benef Microbes (2019) 10:121-135. doi:10.3920/bm2018.0028
8. Kong X-J, Liu K, Zhuang P, Tian R, Liu S, Clairmont C, Lin X, Sherman H, Zhu J, Wang Y, et al. The Effects of Limosilactobacillus reuteri LR-99 Supplementation on Body Mass Index, Social Communication, Fine Motor Function, and Gut Microbiome Composition in Individuals with Prader-Willi Syndrome: a Randomized Double-Blinded Placebo-Controlled Trial. Probiotics Antimicro (2021)1-13. doi:10.1007/s12602-021-09800-9
9. Kong X-J, Wan G, Tian R, Liu S, Liu K, Clairmont C, Lin X, Zhang X, Sherman H, Zhu J, et al. The Effects of Probiotic Supplementation on Anthropometric Growth and Gut Microbiota Composition in Patients With Prader-Willi Syndrome: A Randomized Double-Blinded Placebo-Controlled Trial. Frontiers Nutrition (2021) 8:587974. doi:10.3389/fnut.2021.587974
10. Xiao J, Fiscella K A, Gill S R. Oral microbiome: possible harbinger for children's health. Int J Oral Sci (2020) 12:12. doi:10.1038/s41368-020-0082-x
11. Bantim Y C V, Kussaba S T, Carvalho G P de, Garcia-Junior I R, Roman-Torres C V G. Oral health in patients with Prader-Willi syndrome: current perspectives. Chin Cosmet Investigational Dent (2019) 11:163-170. doi:10.2147/ccide.s183981
12. Olsen I, Yamazaki K. Can oral bacteria affect the microbiome of the gut? J Oral Microbiol (2019) 11:1586422. doi:10.1080/20002297.2019.1586422
13. Fung T C. The microbiota-immune axis as a central mediator of gut-brain communication. Neurobiol Dis (2020) 136:104714. doi:10.1016/j.nbd.2019.104714
14. Galland L. The Gut Microbiome and the Brain. J Med Food (2014) 17:1261-1272. doi:10.1089/jmf.2014.7000
15. Karren B C. A Test Review: Gilliam, J. E. (2014). “Gilliam Autism Rating Scale-Third Edition (GARS-3).” Austin, TX: Pro-Ed. J Psychoeduc Assess 35:342-346. doi:10.1177/0734282916635465
16. McIver L J, Abu-Ali G, Franzosa E A, Schwager R, Morgan X C, Waldron L, Segata N, Huttenhower C. bioBakery: A meta'omic analysis environment. Bioinform Oxf Engl (2017) 34:1235-1237. doi:10.1093/bioinformatics/btx754
17. Rognes T, Flouri T, Nichols B, Quince C, Mahé F. VSEARCH: a versatile open source tool for metagenomics. Peerj (2016) 4:e2584. doi:10.7717/peerj.2584
18. DeSantis T Z, Hugenholtz P, Larsen N, Rojas M, Brodie E L, Keller K, Huber T, Dalevi D, Hu P, Andersen G L. Greengenes, a Chimera-Checked 16S rRNA Gene Database and Workbench Compatible with ARB. Appl Environ Microb (2006) 72:5069-5072. doi:10.1128/aem.03006-05
19. Mallick H, Rahnavard A, McIver L J, Ma S, Zhang Y, Nguyen L H, Tickle T L, Weingart G, Ren B, Schwager E H, et al. Multivariable Association Discovery in Population-scale Meta-omics Studies. Biorxiv (2021)2021.01.20.427420. doi:10.1101/2021.01.20.427420
20. Nagpal S, Singh R, Yadav D, Mande S S. MetagenoNets: comprehensive inference and meta-insights for microbial correlation networks. Nucleic Acids Res (2020) 48:W572-W579. doi:10.1093/nar/gkaa254
21. Aidy S E, Dinan T G, Cryan J F. Immune modulation of the brain-gut-microbe axis. Front Microbiol (2014) 5:146. doi:10.3389/fmicb.2014.00146
22. Cao X, Liu K, Liu J, Liu Y-W, Xu L, Wang H, Zhu Y, Wang P, Li Z, Wen J, et al. Dysbiotic Gut Microbiota and Dysregulation of Cytokine Profile in Children and Teens With Autism Spectrum Disorder. Front Neurosci-switz (2021) 15:635925. doi:10.3389/fnins.2021.635925
23. Cummings J H, Pomare E W, Branch W J, Naylor C P, Macfarlane G T. Short chain fatty acids in human large intestine, portal, hepatic and venous blood. Gut (1987) 28:1221. doi:10.1136/gut.28.10.1221
24. Poul E L, Loison C, Struyf S, Springael J-Y, Lannoy V, Decobecq M-E, Brezillon S, Dupriez V, Vassart G, Damme J V, et al. Functional Characterization of Human Receptors for Short Chain Fatty Acids and Their Role in Polymorphonuclear Cell Activation*. J Biol Chem (2003) 278:25481-25489. doi:10.1074/jbc.m301403200
25. Brown A J, Goldsworthy S M, Barnes A A, Eilert M M, Tcheang L, Daniels D, Muir A I, Wigglesworth M J, Kinghorn I, Fraser N J, et al. The Orphan G Protein-coupled Receptors GPR41 and GPR43 Are Activated by Propionate and Other Short Chain Carboxylic Acids*. J Biol Chem (2003) 278:11312-11319. doi:10.1074/jbc.m211609200
26. Macia L, Tan J, Vieira A T, Leach K, Stanley D, Luong S, Maruya M, McKenzie C I, Hijikata A, Wong C, et al. Metabolite-sensing receptors GPR43 and GPR109A facilitate dietary fibre-induced gut homeostasis through regulation of the inflammasome. Nat Commun (2015) 6:6734. doi:10.1038/ncomms7734
27. Lu Y, Fan C, Li P, Lu Y, Chang X, Qi K. Short Chain Fatty Acids Prevent High-fat-diet-induced Obesity in Mice by Regulating G Protein-coupled Receptors and Gut Microbiota. Sci Rep-uk (2016) 6:37589. doi:10.1038/srep37589
28. Ang Z, Ding J L. GPR41 and GPR43 in Obesity and Inflammation—Protective or Causative? Front Immunol (2016) 7:28. doi:10.3389/fimmu.2016.00028
29. Qin J, Li Y, Cai Z, Li S, Zhu J, Zhang F, Liang S, Zhang W, Guan Y, Shen D, et al. A metagenome-wide association study of gut microbiota in type 2 diabetes. Nature (2012) 490:55-60. doi:10.1038/nature 11450
30. Chaudhari D S, Dhotre D P, Agarwal D M, Gaike A H, Bhalerao D, Jadhav P, Mongad D, Lubree H, Sinkar V P, Patil U K, et al. Gut, oral and skin microbiome of Indian patrilineal families reveal perceptible association with age. Sci Rep-uk (2020) 10:5685. doi:10.1038/s41598-020-62195-5
31. Acheson K J, Zahorska-Markiewicz B, Pittet P, Anantharaman K, Jéquier E. Caffeine and coffee: their influence on metabolic rate and substrate utilization in normal weight and obese individuals. Am J Clin Nutrition (1980) 33:989-997. doi: 10.1093/ajcn/33.5.989
32. Tabrini R, Saneei P, Lankarani K B, Akbari M, Kolahdooz F, Esmaillzadeh A, Nadi-Ravandi S, Mazoochi M, Asemi Z. The effects of caffeine intake on weight loss: a systematic review and dos-response meta-analysis of randomized controlled trials. Crit Rev Food Sci (2018) 59:1-9. doi:10.1080/10408398.2018.1507996
33. Cross B W, Ruhl S. Glycan recognition at the saliva—oral microbiome interface. Cell Immunol (2018) 333:19-33. doi:10.1016/j.cellimm.2018.08.008
34. Rubinoff A B, Latner P A, Pasut L A. Vitamin C and oral health. J Can Dent Assoc (1989) 55:705-7.
35. Ramesh A, Walker S A, Hood D B, Guillén M D, Schneider K, Weyand E H. Bioavailability and Risk Assessment of Orally Ingested Polycyclic Aromatic Hydrocarbons. Int J Toxicol (2004) 23:301-333. doi:10.1080/10915810490517063
36. Wiele T V de, Vanhaecke L, Boeckaert C, Peru K, Headley J, Verstraete W, Siciliano S. Human Colon Microbiota Transform Polycyclic Aromatic Hydrocarbons to Estrogenic Metabolites. Environ Health Persp (2005) 113:6-10. doi:10.1289/ehp.7259
37. Kong X, Liu J, Cetinbas M, Sadreyev R, Koh M, Huang H, Adeseye A, He P, Zhu J, Russell H, et al. New and Preliminary Evidence on Altered Oral and Gut Microbiota in Individuals with Autism Spectrum Disorder (ASD): Implications for ASD Diagnosis and Subtyping Based on Microbial Biomarkers. Nutrients (2019) 11:2128. doi:10.3390/nu11092128
38. Maitre Y, Micheneau P, Delpierre A, Mahalli R, Guerin M, Amador G, Denis F. Did the Brain and Oral Microbiota Talk to Each Other? A Review of the Literature. J Clin Medicine (2020) 9:3876. doi:10.3390/jcm9123876
39. You M, Mo S, Leung W K, Watt R M. Comparative analysis of oral treponemes associated with periodontal health and disease. Bmc Infect Dis (2013) 13:174. doi:10.1186/1471-2334-13-174
40. Zhu B, Macleod L C, Newsome E, Liu J, Xu P. Aggregatibacter actinomycetemcomitans mediates protection of Porphyromonas gingivalis from Streptococcus sanguinis hydrogen peroxide production in multi-species biofilms. Sci Rep-uk (2019) 9:4944. doi:10.1038/s41598-019-41467-9
41. Vonaesch P, Morien E, Andrianonimiadana L, Sanke H, Mbecko J-R, Huus K E, Naharimanananirina T, Gondje B P, Nigatoloum S N, Vondo S S, et al. Stunted childhood growth is associated with decompartmentalization of the gastrointestinal tract and overgrowth of oropharyngeal taxa. Proc National Acad Sci (2018) 115:201806573. doi:10.1073/pnas.1806573115

1. A method to alter the salivary microbiota of a subject in need thereof, the method comprising: orally administering a probiotic composition to the subject.

2. The method of claim 1, wherein the probiotic composition comprises Bifidobacterium animalis subsp. lactis (B. lactis).

3. The method of claim 1, wherein the probiotic composition comprises BL-11.

4. The method of claim 1, wherein the subject in need thereof comprises a subject diagnosed with PWS.

5. The method of claim 1, wherein the probiotic composition is administered for 12 weeks or more.

6. The method of claim 1, wherein the altered salivary microbiota of the subject comprises an increase in α-diversity after 12 weeks of probiotic administration as compared to a control subject or as compared to the subject prior to treatment.

7. The method of claim 1, wherein the altered salivary microbiota of the subject comprises the presence or an increase in the amount of one or more genera selected from Faecalibacterium, Paracoccus, Leptotrichia and Bifidobacterium after 12 weeks of probiotic administration, as compared to an untreated control, or as compared to the subject prior to treatment.

8. The method of claim 1, wherein the altered microbiota of the subject comprises the presence or an increase in the amount one or more of genera selected from Gemella, Aggregatibacter, Corynebacterium, Fusobacterium, and Treponema after 12 weeks of probiotic administration as compared to an untreated control, or as compared to the subject prior to treatment.

9. The method of claim 8, wherein the presence or increase in one or more genera is correlated with increased height of the subject.

10. The method of claim 1, wherein the altered microbiota of the subject comprises one or more of Neisseria, Gemella and Paracoccus after 12 weeks of probiotic administration as compared to an untreated control.

11. The method of claim 10, wherein the presence or increase in one or more genera is correlated with improved social behaviors of the subject.

12. A method of determining efficacy of a probiotic composition to treat PWS in a subject in need thereof, the method comprising: evaluating the salivary microbiota of the subject.

13. The method of claim 12, wherein evaluation comprises determining the presence or relative amount of one or more genera selected from: Faecalibacterium, Paracoccus, Leptotrichia, Bifidobacterium, Gemella, Aggregatibacter, Corynebacterium, Fusobacterium, Treponema, and Neisseria.

14. The method of claim 13, wherein the evaluation occurs prior to probiotic treatment and 12 weeks after probiotic treatment.

15. The method of claim 13, wherein the evaluation comprises comparing the presence or relative amount to a control.

16. The method of claim 15, wherein the control comprise the salivary microbiota of the subject prior to treatment.

17. The method of claim 15, wherein the control comprises the salivary microbiota of an untreated control subject.

18. A kit comprising:
a) a probiotic composition for oral administration;
b) a detection molecule for the detection of one or more of Faecalibacterium, Paracoccus, Leptotrichia, Bifidobacterium, Gemella, Aggregatibacter, Corynebacterium, Fusobacterium, Treponema, and Neisseria.

19. The kit of claim 18, wherein the probiotic composition comprise BL-11.

20. The kit of claim 18, wherein the detection molecule comprises an antibody and/or a nucleic acid.
